(19) United States
(12) Patent Application Publication
(10) Pub. No.: US 2009/0299485 A1
(43) Pub. Date: Dec. 3, 2009

(54) Implant

(76) Inventors: Emmanuel Michelinakis, Athens (GR); Mathios Michelinakis, Athens (GR)

Correspondence Address: GRADY K. BERGEN, 3333 LEE PARKWAY, SUITE 600, DALLAS, TX, 75219, US

(21) Appl. No.: 12/128,000

(22) Filed: May 28, 2008

(51) Int. Cl. A61F 2/36; A61F 2/28

(52) U.S. Cl. 623/23.36; 623/23.15; 623/16.11

(57) ABSTRACT

The invention provides a stem implant device typically for use to be received in a hip as part of hip replacement apparatus. The implant has a tapered design and includes an intermediate portion with a surface finish which encourages bone in-growth and a plurality of groups of rib members which increases the stability of the implant in the host bone without pain and enhance long term fixation.

### BACKGROUND

The invention to which this application relates is to an improvement in the form of a stem implant which is for use, typically as part of a hip replacement system.

In any hip replacement operation and in subsequent use of the hip replacement, there are several factors which need to be taken into account. One of these is the longevity of the implant once it has been fitted and also the comfort of the patient to whom the implant has been fitted and the ability for the hip replacement to be used in natural range of motion. In the initial stages, it is important that there is provided a strong fixation of the implant in position in the patient. It is found that if a strong fixation can be achieved initially then this improves the chances of long term stability of the implant and as a result the improved quality of life for the patient can be achieved.

The use of stem implants is well known and generally known to comprise a neck or spigot, leading to an elongate member with a distal tip. The elongate member is fitted into the femur bone and the neck or spigot receives a femoral head component thereon. There are many different designs and variations of the implant which are the subject of many differing prior art patents. However, even with these different designs, problems are still experienced in both the initial fixation and long term stability of the implants in the patient. These problems, in the worst cases, can cause considerable pain and discomfort to the patient, can cause failure of the implant itself and/or require further surgery to be performed for the removal of the prosthesis. These requirements add further expense to the medical system and cause significant distress to the patient. The stem is implanted directly into the femur bone press-fit without the use of cement. Fixation is defined as the stable mechanical joining of the femoral component to the femur such that no relative motion occurs between the implant and the bone at the fixation interface. Tapered uncemented femoral components rely on initial three-point fixation followed by proximal porous bone ingrowth or ongrowth for continued stability. The points of fixation are achieved by the implant at two spaced points on the posterior side of the elongate member or stem and at an intermediate location on the anterior side.

### SUMMARY

An aim of the present invention is to provide a stem implant device in a form which provides improved fixation characteristics when initially fitted in the patient, and, as a result, improved stability and longevity of the hip implant.

In a first aspect of the invention, there is provided an implant, said implant having, at a first end, a neck formation, leading to an elongate member, the distal end of which defines the opposing end of the implant, said implant having, at least at a portion intermediate or proximal of the neck formation and the distal end, a surface finish which is created by the application of a coating material thereto.

In one embodiment at a portion leading from the said portion towards the distal end, there are provided at least two groups of rib members, each group having a plurality of rib members.

Typically, the surface finish of the said portion is provided by applying a porous coating thereto. The porous coating is provided to encourage bone in-growth onto the implant and thus provides improved fixation and long term stability.

In one embodiment, the porous coating is achieved by applying titanium beads to promote the growth of the host bone onto the surface of the implant. The coating may be applied around the circumference of the said intermediate, also referred to as the proximal part of the stem portion so that the whole of the portion has a surface finish formed by the porous coating.

In one embodiment the area of the implant between the porous coated intermediate part and the distal end is grit blasted to encourage bone attachment and on-growth thereon. In one embodiment the distal end has a tip which is polished.

In one embodiment, the longitudinal axes of the rib members are substantially parallel with the longitudinal axis of the elongate member. Typically the rib members in each group are spaced apart along the circumference of the elongate member.

In one embodiment two groups of rib members are provided, said groups spaced so as to be positioned on opposing sides of the elongate member. Typically each rib member in each group has a first end in line with the first end of each of the other rib members in the group but the respective lengths of the rib members vary depending on the position of the rib member in the group.

In one embodiment the height of the top of the rib member from the implant surface decreases from a first location at the end of the rib closest to the neck, to the opposing end. Typically the decrease in height is continuous along the length of said rib. In one embodiment the width of each rib remains constant along part of the length of the rib and from which point the width of the rib decreases towards a point at the end of the rib closest to the distal end of the elongate member.

Typically the longitudinal axis of each of the ribs in each group remain substantially parallel.

Typically, the implant is manufactured from titanium and may be a Titanium 6 Aluminium 4 Vanadium (Ti6Al4V) alloy Thus, typically, with the use of the titanium beads coating in the intermediate portion the external surface of the implant is formed of titanium.

Typically, the elongate member, from the intermediate portion to the distal end, is tapered and may be tapered by 3 degrees and typically the tapering is in the three planes of coronal, sagittal and transverse. This allows the elongate member to match anatomical characteristics of the femur bone and is found to prevent stress and provides correct anatomical loading distribution throughout the femur hence reducing distal fixation which may cause thigh pain in the patient to which the implant is fitted.

Furthermore, the taper design of the component acts as a wedge to physically lock the implant into place, proximally, and to prevent the implant from sinking deeper into the femur than is required. The wedge shape provides a frictionally stable implant in the femur enhancing the long term stability of the prosthesis.

In a further aspect of the invention there is provided a method for forming an implant, said method comprising forming the implant to have, at a first end, a neck formation, leading to an elongate member, the distal end of which defines the opposing end of the implant, said elongate member including at least two groups of rib members formed thereon and characterized in that once formed, at least at a portion intermediate or proximal of the neck formation and the distal end, a coating material of beads is applied thereto to provide a porous surface finish thereon.

Typically the beads are of titanium and are provided to encourage bone in growth in said coated area.

In one embodiment the beads are applied onto the surface of the implant at the required location thereon and the implant is heated, at least at the said location, to a sufficient temperature to connect the beads to each other and to the implant.

In one embodiment the method includes the further step of grit blasting at least the area of the elongate member between the said portion and the distal end to encourage rough surface finish to encourage bone on growth.

In a further embodiment the method includes the step of polishing the tip at the distal end.

In one embodiment the implant is formed, typically by forging, of titanium.

### BRIEF DESCRIPTION OF THE DRAWINGS

For a more complete understanding of the present invention, and the advantages thereof, reference is now made to the following descriptions taken in conjunction with the accompanying figures, in which:

FIGS. 1a-1c illustrate a plan, elevation and side view of a stem implant in accordance with one embodiment of the invention;

FIGS. 2a-2p illustrate cross sectional views of the implant of FIG. 1;

FIGS. 3a-3o illustrate cross sectional views of the implant of FIG. 1 but of different parts than shown in FIGS. 2a-2p;

FIG. 4 illustrates a cross sectional view along line F-F of FIG. 2b, showing one half of the implant and ribs in greater detail; and

FIG. 5 illustrates an embodiment of an implant in accordance with the invention.

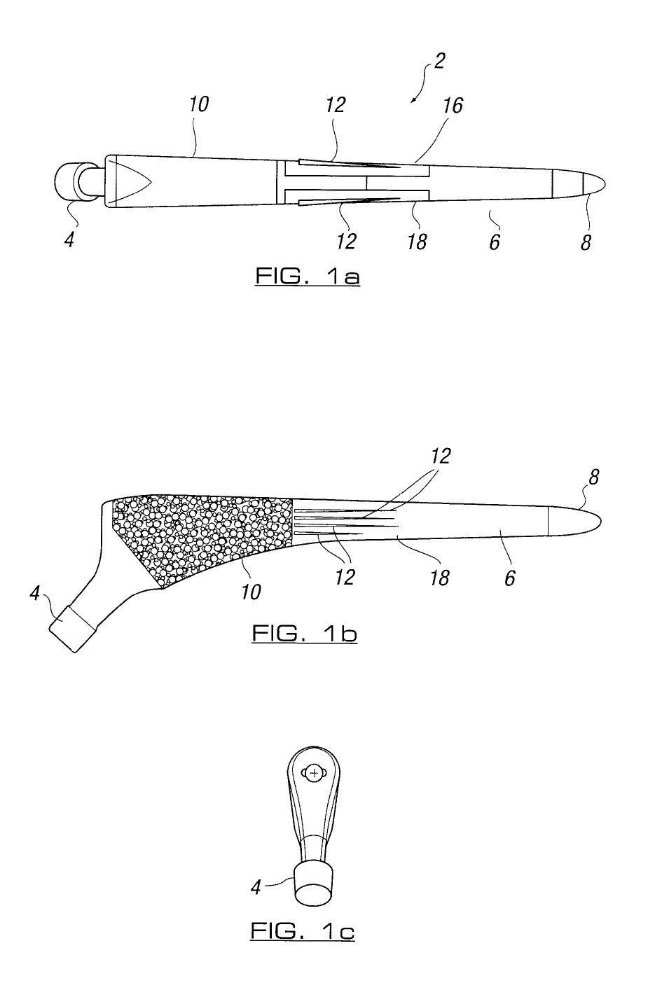

### DETAILED DESCRIPTION

Referring to the drawings there is shown a stem implant 2 of a design in accordance with one embodiment of the invention. The implant comprises a neck portion 4 leading to an elongate member section 6 with a distal tip 8. The neck portion 4 includes a part which is provided to receive a head thereon which is not shown but which can be of any conventional form. Following from the neck portion there is provided an intermediate portion 10 which can also be referred to as the proximal of a stem portion. In accordance with a preferred embodiment of the invention, and as shown, this intermediate portion 10 is coated with a porous material. It is also coated around the entire intermediate portion. In the embodiment shown the coating which is applied is sintered titanium beads and the pore size is, in one embodiment, in the range of 80-259 μm. In one embodiment the microsphere porosity is in the range of 35-40%. It is found that the provision of this coating improves the ongrowth of bone once the implant has been fitted in position and, as a result increases the stability and strength of the implant when in position.

The elongate member 8 is also provided with a plurality of rib members 12. The rib members are provided in a specific configuration which has been found to improve the fixation of the stem implant in the femur and in particular prevent rotation of the implant. The elongate member also tapers inwardly towards the distal tip 8 at a taper of 3 degrees which is found to match closely with the taper of the femur and hence improve the comfort of the patient with the implant fitted.

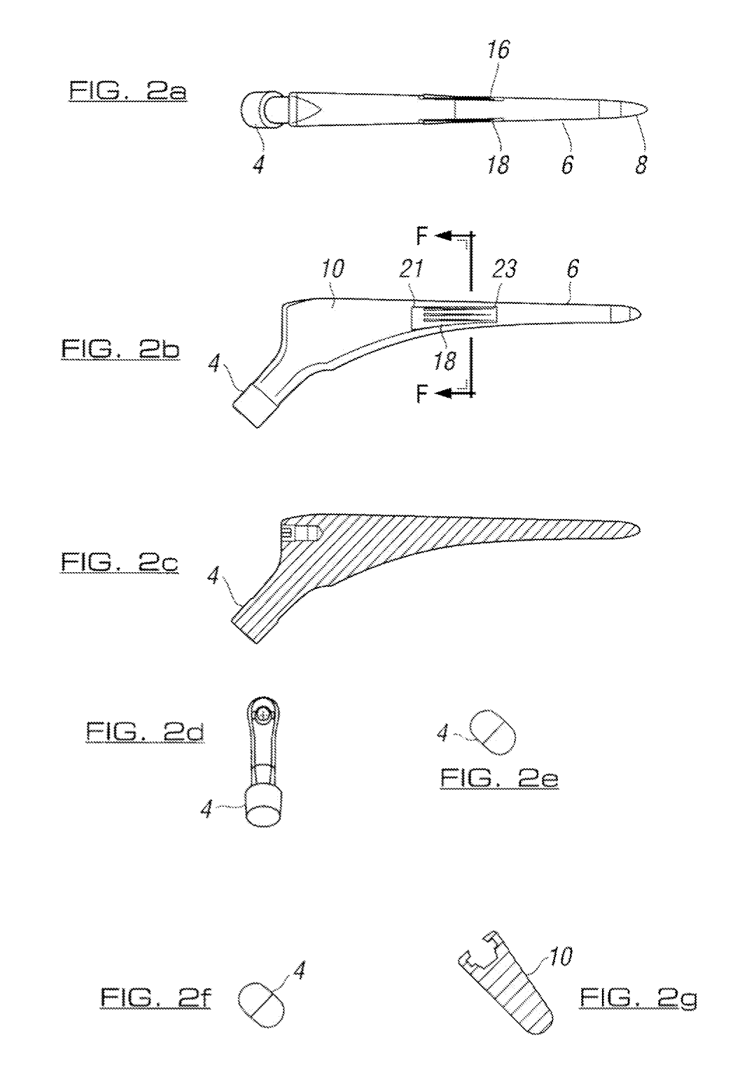

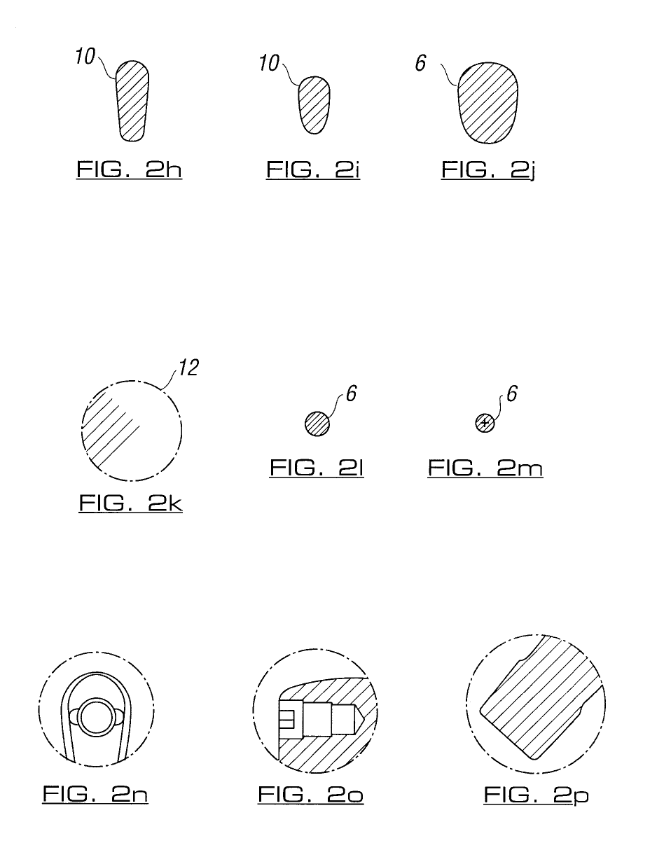

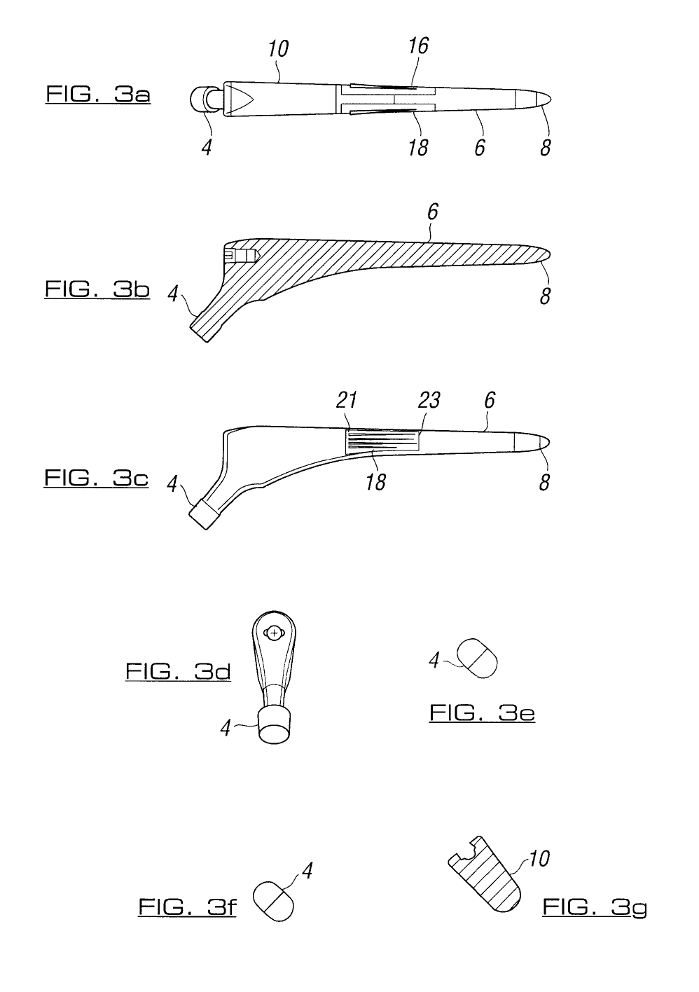

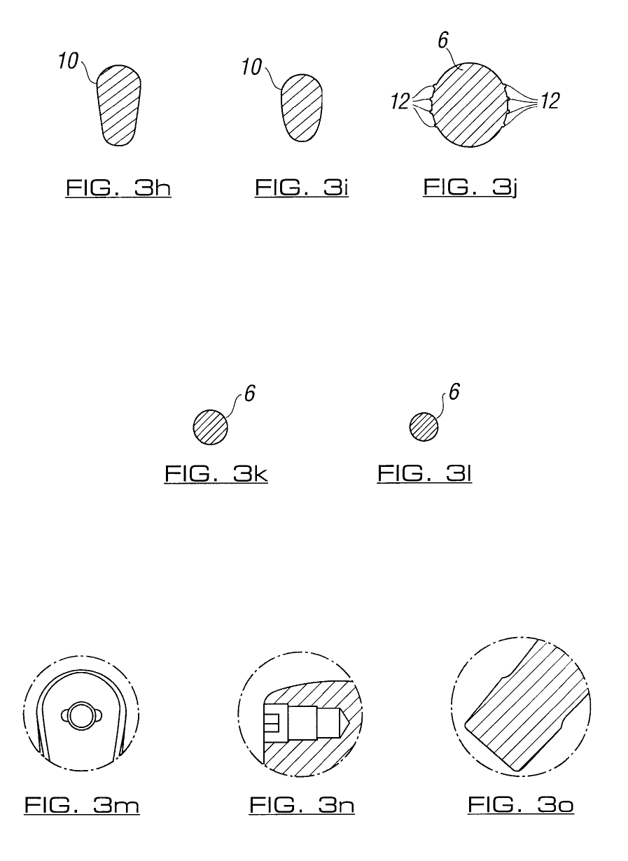

It will be seen from FIGS. 2a-2p and 3a-3o that the rib members 12 are provided in two groups 16, 18 and neither group extends along the length to the distal end, which is contrary to other conventional stem implants. Instead, in accordance with the invention, the ribs extend from the intermediate coated portion towards the distal tip for a length of approximately a third to half of the length of the elongate member. Each rib narrows in the direction of the distal tip and tapers to a point. In one embodiment the spacing between the centres of adjacent ribs in each group is 2.7 mm.

In FIGS. 2a-2p the embodiment shown has three ribs provided in each of the groups on opposing sides of the implant. In FIGS. 3a-3o four rib members are provided in each group.

In whichever embodiment, typically each of the ribs decreases in height from the rib end 21 towards the rib end 23. While the ribs may be of different lengths, typically in each case the decrease in height for each particular rib will be continuous. The width of each of the ribs may be kept constant for a substantial length of the same until at a location along the length, intermediate the ends, the rib width is narrowed such that at the end 23 the rib narrows to a point.

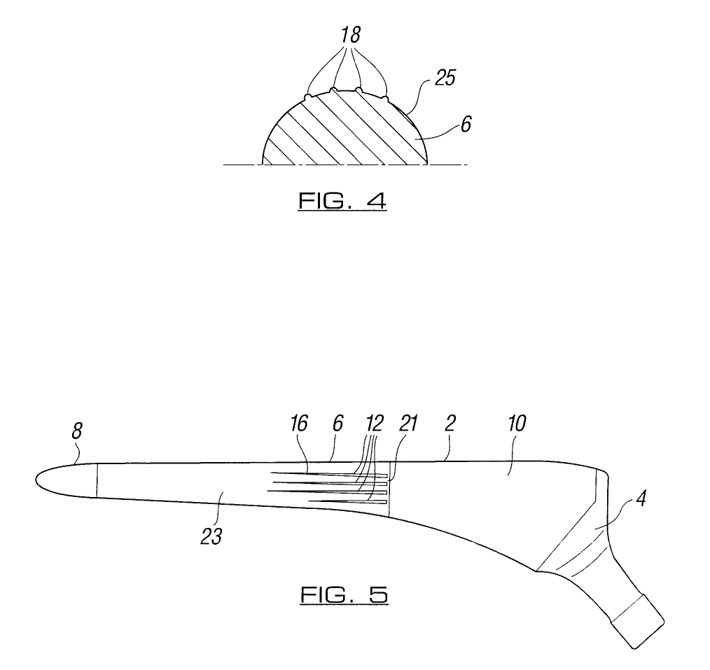

FIG. 4 shows a cross section through one half of the implant along the line F-F of FIG. 2b, of larger scale. It shows how each of the ribs in the group 18 are spaced apart by a set distance and located so as to lie along the shaped outer periphery wall of the implant 6.

FIG. 5 illustrates one embodiment of an implant in accordance with the invention. The provision of one of groups of rib members can be seen with, in this embodiment four rib members, of varying lengths being provided in the group. Also clearly shown is the intermediate portion with a surface finish created by the application of the beads thereto. The beads can be applied to the required location on the implant, possibly using a mixture of the beads with adhesive or resin medium. The implant and beads are then heated to a sufficient temperature to allow the same to bond to form the coating. The area of the elongate member from the said portion towards the distal tip and in which the rib members are located, is grit blasted to form a roughened surface in comparison to the polished distal tip.

The provision of the ribs and the placing of the same in groups in conjunction with the roughened surface finish created by grit blasting the area of the implant in which the rib members are located is found to improve the fixation of the stem implant in position. This, in conjunction with the improved bone ongrowth which is achieved by coating the entire surface of the intermediate portion 10, means that the length of the elongate member or stem formed in accordance with the invention can be significantly reduced in comparison to conventional stem implants as the required level of anchoring of the implant in position can be achieved with the implant in accordance with the invention. The reduction in length means that the distal tip does not have to be placed as far into the patient's femur and, therefore, there isn't any possibility of bone formation due to mechanical stress, distally, which may cause distal fixation of the implant, and thigh pain. It also reduces the surgical invasive work required and in turn reduces discomfort to the patient.

While the invention has been shown in only some of its forms, it should be apparent to those skilled in the art that it is not so limited, but is susceptible to various changes and modifications without departing from the scope of the invention. Accordingly, it is appropriate that the appended claims be construed broadly and in a manner consistent with the scope of the invention.

We claim:

1. An implant, said implant having, at a first end, a neck formation, leading to an elongate member, the distal end of which defines the opposing end of the implant, said implant having, at least at a portion intermediate, or proximal of the neck formation and the distal end, a surface finish which is created by the application of a coating material thereto.

2. An implant according to claim 1 characterized in that at a portion leading from the said intermediate portion towards the distal end, there are provided at least two groups of rib members, each group having a plurality of rib members.

3. An implant according to claim 1 characterized in that the surface finish of the intermediate portion is provided by applying a porous coating material thereto.

4. An implant according to claim 3 characterized in that the porous coating is provided to encourage bone growth into the implant.

5. An implant according to claim 1 characterized in that the porous coating material includes titanium beads.

6. An implant according to claim 1 characterized in that the coating is applied around the circumference of the said intermediate portion so that the whole of the portion has a surface finish formed by the porous coating.

7. An implant according to claim 1 characterized in that the longitudinal axes of the rib member are substantially parallel with the longitudinal axis of the elongate member.

8. An implant according to claim 7 where the rib members in each group are spaced apart about the circumference of the elongate member.

9. An implant according to claim 1 characterized in that two groups of rib members are provided, said groups positioned on opposing sides of the elongate member.

10. An implant according to claim 9 characterized in that each rib member in each group has a first end in line with the first end of each of the other rib members in the group but the respective lengths of the rib members in the group varies.

11. An implant according to claim 10 characterized in that the height of the rib members in each group decrease from the rib member closest to the neck, to the rib member at the opposing end of the group.

12. An implant according to claim 11 characterized in that the decrease in height is continuous in said direction away from the first location.

13. An implant according to claim 12 characterized in that the width of each rib member is substantially constant along part of the length of the rib member and from which point, the width of the rib decreases towards the point at the end of the rib member closest to the distal end of the elongate member.

14. An implant according to claim 1 characterized in that the longitudinal axes of each of the ribs in each group remain substantially parallel.

15. An implant according to claim 1 characterized in that the implant is manufactured from titanium.

16. An implant according to claim 15 characterized in that the external surface of the implant, with the exception of the intermediate portion, is formed of titanium.

17. An implant according to claim 16 characterized in that the said surface is polished.

18. An implant according to claim 1 characterized in that the elongate member, from the intermediate portion to the distal end is tapered.

19. An implant according to claim 18 characterized in that the tapering is in three planes of coronal, sagittal and transverse.

20. An implant according to claim 18 wherein the taper angle is 3 degrees.

21. An implant according to claim 1 characterized in that the external surface of the intermediate portion is formed of titanium beads.

22. An implant according to claim 1 characterized in that the external surface of the portion between the intermediate and the distal end is grit blasted to form a relatively rough surface finish.

23. A method for forming an implant, said method comprising forming the implant to have, at a first end, a neck formation, leading to an elongate member, the distal end of which defines the opposing end of the implant, said elongate member including at least two groups of rib members formed thereon and characterized in that once formed at least at a portion intermediate or proximal of the neck formation and the distal end, a coating material of beads is applied thereto to provide a porous surface finish thereon.

24. A method according to claim 23 characterized in that the method includes the step of grit blasting at least the area of the elongate member between the said portion and the distal end.

25. A method according to claim 23 characterized in that the method includes the step of polishing the tip at the distal end.

26. A method according to claim 23 characterized in that the implant is formed from titanium.

27. A method according to claim 26 characterized in that the implant is forged.
